(19) United States
(12) Patent Application Publication
(10) Pub. No.: US 2026/0242847 A1
(43) Pub. Date: Aug. 20, 2026

(54) METHOD OF DETECTING TARGET SUBSTANCE, REAGENT, AND IN VITRO DIAGNOSTIC KIT

(71) Applicant: CANON KABUSHIKI KAISHA, Tokyo (JP)

(72) Inventors: FUMIO YAMAUCHI, Kanagawa (JP); NORISHIGE KAKEGAWA, Tokyo (JP); KENJI KITAGAWA, Shiga (JP); TSUYOSHI NOMOTO, Tokyo (JP)

(21) Appl. No.: 19/545,441

(22) Filed: Feb. 20, 2026

(30) Foreign Application Priority Data
Feb. 20, 2025 (JP) 2025-026281

(51) Int. Cl. C12Q 1/52; G01N 21/64

(52) U.S. Cl. CPC C12Q 1/52; G01N 21/6428; G01N 2021/6439

(57) ABSTRACT

Provided are a fast, simple, high-sensitivity method of detecting a target substance related to an enzyme reaction of a transglutaminase in a liquid sample, a reagent, and an in vitro diagnostic kit. Specifically, provided is a reagent for detecting a transglutaminase through use of a value related to fluorescence anisotropy, the reagent including a plurality of luminescent particles each having a first substrate of the transglutaminase.

### BACKGROUND

### Field of the Technology

The present disclosure relates to a method of detecting a target substance, a reagent, and an in vitro diagnostic kit.

### Description of the Related Art

Enzymes each have a role in catalyzing a chemical reaction in a living body, and the measurement of enzyme activity is extremely important in the fields of not only medical research of enzymes but also inspection of enzymes, clinical inspections utilizing enzymes as reagents, and substance production utilizing enzymes.

A transglutaminase (hereinafter sometimes abbreviated as “TG”) is an enzyme, which uses a glutamine residue in a polypeptide as a substrate, and catalyzes an amide bond-forming reaction between a carboxamide side chain of the glutamine residue and an amino group from a lysine residue, for example. The family of the transglutaminases is formed of 8 enzymes/enzyme precursors referred to as “TG1” to “TG7” and “factor XIII.” It has been known that the transglutaminase has a physiological role, such as blood coagulation by the factor XIII or localized epidermization by the transglutaminase. In recent years, the pathological importance of the transglutaminase in a neurodegenerative disease has been proven, and hence the development of a high-sensitivity measurement technology for transglutaminase has been expected in order to perform drug development, treatment, or diagnosis based on the transglutaminase.

Fluorescence polarization measurement has been known as one of the methods of measuring enzyme activity. The fluorescence polarization measurement, which utilizes fluorescence polarization, is a method of measuring the rotational motion of a fluorescent substance. When the fluorescent substance is excited, the anisotropy of its fluorescence (also referred to as “degree of fluorescence polarization”) changes in accordance with the rotational motion of the fluorescent substance. That is, the method utilizes the fact that when the fluorescent substance does not rotate, polarized luminescence is observed, whereas when the fluorescent substance freely rotates, fluorescence is radiated in all planes, and as a result, the polarization is eliminated. A feature of the fluorescence polarization measurement is that in the assay, the degree of fluorescence polarization is used as an indicator, not the luminescence intensity of the fluorescence. Another feature is that the method does not require a separation or washing operation. That is, a homogeneous assay is possible, and a specimen can be added to a solution and subjected to measurement as is. Thus, no complicated separation work is required, and measurement time can be shortened.

A method of measuring the activity of a transglutaminase with fluorescence has heretofore been disclosed. In International Publication No. WO2018/004014, the activity of a transglutaminase is examined by measuring fluorescence resonance energy transfer between different fluorescent dyes caused by the bonding of two kinds of substrate peptides labeled with the fluorescent dyes based on the activity of the transglutaminase. However, in the measurement method disclosed in the above-mentioned literature, one substrate peptide molecule is labeled with one fluorescent molecule, and hence a signal change amount per one reaction reduces. Accordingly, when the concentration of an enzyme is extremely low, it has become difficult to measure the activity of the enzyme in some cases. In addition, the fluorescent dyes are each susceptible to its surrounding environment, and hence in a liquid sample containing a large amount of contaminants such as blood, the fluorescent dyes each adsorb to, for example, a protein or a lipid serving as a contaminant to reduce the substrate recognizability of the peptide bonded to the fluorescent dye. Accordingly, it has become difficult to perform high-sensitivity measurement in some cases.

U.S. Patent Application Publication No. 2024/0093087 discloses an immunoassay technology using fluorescence polarization measurement, and a high-sensitivity immunoassay is constructed by using luminescent particles to aggregate the luminescent particles via an antigen-antibody reaction, and by observing a change in degree of fluorescence polarization before and after the aggregation. However, in the measurement method disclosed in the above-mentioned literature, it is extremely difficult to measure enzyme activity because the antigen-antibody reaction is used.

### SUMMARY

The present disclosure provides that a value related to the fluorescence anisotropy of the amount or activity of an enzyme or a substance related to an enzyme reaction can be obtained in a short time period and with high sensitivity by introducing a mechanism that causes the interparticle aggregation of luminescent particles through the action of a transglutaminase in a liquid sample to greatly reduce the mobility of the luminescent particle.

That is, according to one aspect of the present disclosure, there is provided a reagent for detecting a transglutaminase through use of a value related to fluorescence anisotropy, the reagent including a plurality of luminescent particles each having a first substrate of the transglutaminase.

In addition, according to another aspect of the present disclosure, there is provided a reagent for detecting one of an activator of a transglutaminase, an inhibitor of the transglutaminase, or a second substrate of the transglutaminase through use of a value related to fluorescence anisotropy, the reagent including: the transglutaminase; and a plurality of luminescent particles each having a first substrate of the transglutaminase.

In addition, according to another aspect of the present disclosure, there is provided an in vitro diagnostic kit including any one of the above-mentioned reagents.

In addition, according to another aspect of the present disclosure, there is provided a method of detecting a target substance related to an enzyme reaction of a transglutaminase in a liquid sample, the method including: forming, in the liquid sample containing the transglutaminase and a plurality of luminescent particles each having a first substrate of the transglutaminase, a crosslinked structure of the first substrate of the transglutaminase that the plurality of luminescent particles each have based on activity of the transglutaminase; and obtaining a value related to fluorescence anisotropy of the liquid sample after the first step.

Features of the present disclosure will become apparent from the following description of embodiments with reference to the attached drawings. The following description of embodiments is described by way of example.

### BRIEF DESCRIPTION OF THE DRAWINGS

FIG. 1 is a flowchart providing a method of detecting a target substance according to one aspect of the present disclosure.

FIG. 2 is a schematic diagram for describing a high-sensitivity method of detecting a transglutaminase according to one aspect of an embodiment of the present disclosure.

FIG. 3 is a schematic diagram for describing a method of detecting the inhibitor of a transglutaminase according to one aspect of the embodiment of the present disclosure.

FIG. 4 is a schematic diagram for describing a method of detecting the second substrate of a transglutaminase according to one aspect of the embodiment of the present disclosure.

### DESCRIPTION OF THE EMBODIMENTS

The present disclosure is described in more detail below. The term “detection” as used in the present disclosure is used in a meaning including each of the following cases: to determine the presence or absence of a target substance; to detect the target substance; and to qualitatively and quantitatively determine the amount and activity of the target substance.

### (Value Related to Fluorescence Anisotropy Determined by Fluorescence Polarization Measurement)

The present disclosure is directed to a method of detecting the amount and activity of a substance related to the enzyme reaction of a transglutaminase, such as the transglutaminase, the second substrate of the transglutaminase, the activator of the transglutaminase, or the inhibitor of the transglutaminase, through use of a luminescent particle by obtaining a value related to its fluorescence anisotropy.

Fluorescence polarization measurement is used to analyze the mobility of a fluorescent molecule in a solution. The principle of the fluorescence polarization measurement is described by the case of the luminescent particle of the present disclosure. When a luminescent particle (particle containing a luminescent molecule exhibiting fluorescence anisotropy) in a liquid is excited by plane-polarized light, the particle emits polarized fluorescence in the same plane. However, when the luminescent particle is rotated by its Brownian motion during the excited state, the particle emits fluorescence to a plane different from the excitation plane, and hence its fluorescence anisotropy is eliminated. In other words, the value related to the fluorescence anisotropy represents the degree of rotational motion of the luminescent particle from being excited to emitting fluorescence.

When the luminescent particle in the liquid is singly dispersed in the solution, the particle shows low fluorescence anisotropy because the particle is vigorously rotated by its Brownian motion. Meanwhile, when the luminescent particles aggregate, the Brownian motion in the solution reduces to increase the fluorescence anisotropy. Accordingly, in the fluorescence polarization measurement, the mobility of the luminescent particle in the solution is analyzed by using a change in fluorescence anisotropy thereof as an indicator. A unit “milli P” (hereinafter abbreviated as “mp”) representing a change in plane polarization may be used as an indicator of the fluorescence anisotropy.

### (Method of Detecting Target Substance)

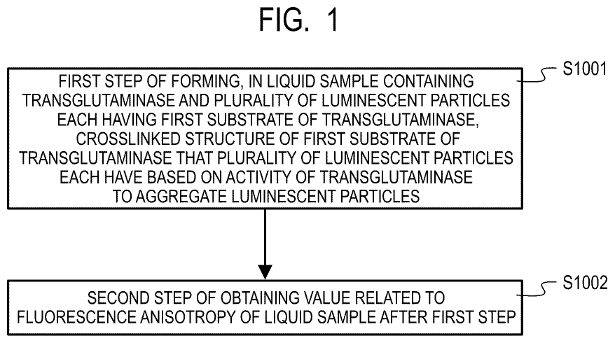

A method of detecting a target substance of the present disclosure is a method of detecting a target substance related to the enzyme reaction of a transglutaminase in a liquid sample, and includes the following steps (1) and (2) as illustrated in FIG. 1. The method of detecting a target substance of the present disclosure may include the following step (3).
(1) A first step (S1001) of forming, in the liquid sample containing the transglutaminase and a plurality of luminescent particles each having the first substrate of the transglutaminase, a crosslinked structure of the first substrate of the transglutaminase that the plurality of luminescent particles each have based on the activity of the transglutaminase to aggregate the luminescent particles
(2) A second step (S1002) of obtaining a value related to the fluorescence anisotropy of the liquid sample after the first step
(3) A third step of detecting the target substance related to the enzyme reaction of the transglutaminase in the liquid sample based on the value related to the fluorescence anisotropy.

According to the present disclosure, the high-sensitivity method of detecting a target substance related to the enzyme reaction of a transglutaminase in a liquid sample has the following features: (i) the luminescent particle is used, and hence a signal intensity per molecule is enhanced unlike a conventional low-molecular weight substrate; (ii) the mobility of the luminescent particle can be significantly suppressed in a short time period through the action of an enzyme, and hence the value related to the fluorescence anisotropy obtained from the luminescent particle greatly changes in a short time period; and (iii) the measurement of the value related to the fluorescence anisotropy eliminates the need for the separation and washing of a contaminant and an unreacted product, and hence even when the activity of the transglutaminase or a substrate thereof serving as the target substance is low, or the amount thereof is small, the amount and activity of the target substance can be detected with high sensitivity and quickly as compared to an existing method.

### (Measurement Principle 1: Measurement of Transglutaminase Activity)

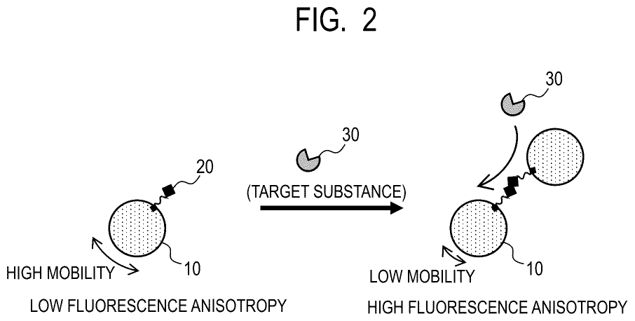

The principle of the present disclosure is described with reference to FIG. 2 by a method of measuring the activity of a transglutaminase.

A transglutaminase 30 serving as a target substance causes the formation of the crosslinked structure of luminescent particles 10 through a first substrate 20 (e.g., glutamine or lysine) of the transglutaminase on each of the luminescent particles 10 by virtue of its activity (catalyzing a reaction for forming an amide bond between a glutamine residue in a polypeptide and an amino group). At this time, the luminescent particles 10 before the addition of the transglutaminase 30 are dispersed in a solution, and hence each have large mobility. However, when the transglutaminase 30 is added, a bond through the first substrate 20 is formed in accordance with its activity to form the crosslinked structure (also referred to as “aggregation”) of the luminescent particles 10, and as a result, the mobility of each of the luminescent particles 10 reduces. The reduction in mobility is observed as an increase in fluorescence anisotropy of the luminescent particle 10. Accordingly, the activity of the transglutaminase 30 in a specimen can be determined by obtaining a value related to the fluorescence anisotropy of each of the luminescent particles 10.

In actuality, the many luminescent particles 10 are present in the solution, and the ratio of the luminescent particles 10 that aggregate increases in accordance with the activity of the transglutaminase 30 in the specimen. Herein, the value related to the fluorescence anisotropy, the value being obtained by fluorescence polarization measurement, is the average of the entirety of the solution, that is, the entirety of the luminescent particles 10, and hence the ratio of the luminescent particles 10 that aggregate and the value related to the fluorescence anisotropy correlate with each other.

For example, the activity of the transglutaminase in the specimen may be determined from the result of the measurement of a value related to the fluorescence anisotropy of the solution based on a calibration curve between a value related to fluorescence anisotropy and activity obtained in advance through use of a transglutaminase whose activity is known.

In the present disclosure, not the activity of the transglutaminase but the amount thereof may be measured. That is, the measurement may be performed by obtaining the value related to the fluorescence anisotropy of the solution dependent on the amount of the transglutaminase while keeping the activity constant.

### (Measurement Principle 2: Measurement of Inhibitor of Transglutaminase)

In the present disclosure, an inhibitor serving as an example of a substance related to the enzyme reaction of a transglutaminase may be subjected to measurement by the same method.

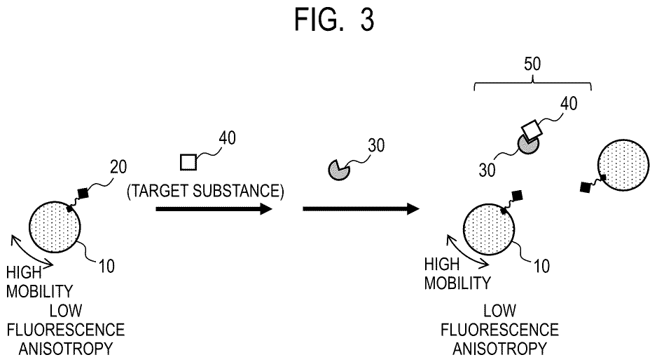

As illustrated in FIG. 3, for example, the inhibition efficiency of an inhibitor 40 of a transglutaminase serving as a target substance on a specified amount of the transglutaminase 30 may be measured by using a value related to the fluorescence anisotropy of the luminescent particle 10 as an indicator.

Specifically, a liquid sample contains a sufficiently large amount of the transglutaminase 30 and/or the transglutaminase 30 having sufficiently high activity. The inhibitor 40 of the transglutaminase serving as the target substance is bonded to the transglutaminase 30 with any affinity to inhibit the activity of the transglutaminase 30. That is, when a transglutaminase 50 having bonded thereto the inhibitor 40 is produced, the activity of the transglutaminase is lost. As a result, the extent to which the luminescent particles 10 are aggregated by the transglutaminase 30 is reduced. When the inhibition efficiency is 100%, the luminescent particles 10 do not aggregate. As a result, the mobility of each of the luminescent particles 10 does not change, and the fluorescence anisotropy of the luminescent particle 10 also does not change. Meanwhile, when the inhibition efficiency is 0%, the luminescent particles 10 aggregate. As a result, the formation of such crosslinked structure of the luminescent particles 10 as illustrated in FIG. 2 occurs to increase the fluorescence anisotropy of each of the luminescent particles 10. That is, a change in value related to the fluorescence anisotropy is observed in accordance with the inhibition efficiency of the inhibitor 40 on the transglutaminase 30. Accordingly, many inhibitor candidates can be simply screened by the detection method of the present disclosure.

### (Measurement Principle 3: Measurement of Activator of Transglutaminase)

In addition to the inhibitor, the activation efficiency of the activator of a transglutaminase may be similarly detected (not shown).

Specifically, a liquid sample contains a small amount of the transglutaminase 30 and/or the transglutaminase 30 having low activity. The activator serving as the target substance is bonded to the transglutaminase 30 with any affinity to improve the activity of the transglutaminase 30. As a result, the extent to which the luminescent particles 10 are aggregated by the transglutaminase 30 can be increased. When the activation efficiency is 0%, the luminescent particles 10 do not aggregate. As a result, the mobility of each of the luminescent particles 10 does not change, and the fluorescence anisotropy of the luminescent particle 10 also does not change. Meanwhile, when the activation efficiency is 100%, the luminescent particles 10 aggregate. As a result, the formation of such crosslinked structure of the luminescent particles 10 as illustrated in FIG. 2 occurs to increase the fluorescence anisotropy of each of the luminescent particles 10. That is, a change in value related to the fluorescence anisotropy is observed in accordance with the activation efficiency of the activator on the transglutaminase 30. Accordingly, many activator candidates can be simply screened by the detection method of the present disclosure.

### (Measurement Principle 4: Measurement of Second Substrate of Transglutaminase)

In addition, the second substrate of a transglutaminase may be subjected to measurement in the same manner as in the inhibition efficiency in the measurement principle 2.

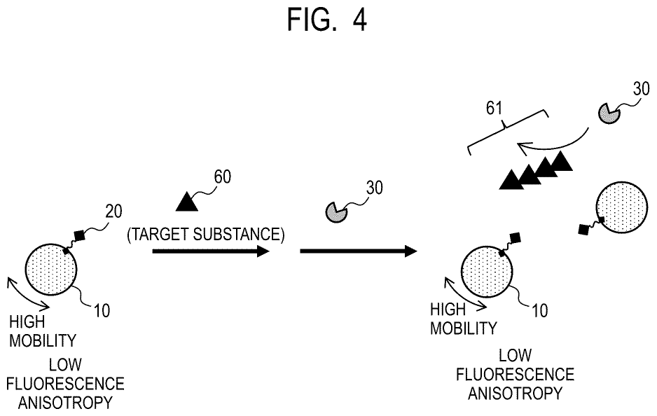

As illustrated in FIG. 4, for example, the inhibition efficiency of a second substrate 60 of a transglutaminase serving as a target substance on a specified amount of the transglutaminase 30 may be measured by using a value related to the fluorescence anisotropy of the luminescent particle 10 as an indicator. The term “inhibition” as used herein means that the second substrate 60 competitively inhibits the reaction of the transglutaminase 30 with the first substrate 20.

Specifically, a liquid sample contains a sufficiently large amount of the transglutaminase 30 and/or the transglutaminase 30 having sufficiently high activity. The second substrate 60 of the transglutaminase serving as the target substance independently can form a crosslinked structure as compared to the first substrate 20 through the activity of the transglutaminase 30 (the second substrate 60 has substrate specificity higher than that of the first substrate 20). That is, when a crosslinked structure 61 of the second substrate 60 is formed, the activity of the transglutaminase 30 on the first substrate 20 is inhibited. As a result, the extent to which the luminescent particles 10 are aggregated by the transglutaminase 30 is reduced. When the inhibition efficiency is 100%, the luminescent particles 10 do not aggregate. As a result, the mobility of each of the luminescent particles 10 does not change, and the fluorescence anisotropy of the luminescent particle 10 also does not change. Meanwhile, when the inhibition efficiency is 0%, the luminescent particles 10 aggregate. As a result, the formation of such crosslinked structure of the luminescent particles 10 as illustrated in FIG. 2 occurs to increase the fluorescence anisotropy of each of the luminescent particles 10. That is, a change in value related to the fluorescence anisotropy is observed in accordance with the inhibition efficiency of the second substrate 60 on the transglutaminase 30. Accordingly, many transglutaminase substrate candidates can be simply screened by the detection method of the present disclosure.

### (Specific Description of Detection Method)

An example of the present disclosure includes the following steps. In this case, the detection method of this embodiment may include the following step (3).
(1) A first step of forming, in the liquid sample containing the transglutaminase and the plurality of luminescent particles each having the first substrate of the transglutaminase, the crosslinked structure of the first substrate of the transglutaminase that the plurality of luminescent particles each have based on the activity of the transglutaminase
(2) A second step of obtaining a value related to the fluorescence anisotropy of the liquid sample after the first step
(3) A third step of detecting the target substance related to the enzyme reaction of the transglutaminase in the liquid sample based on the value related to the fluorescence anisotropy.

Each step is described in more detail below.

### (First Step)

In the present disclosure, the first step is a step of forming a crosslinked structure (also referred to as “aggregation”) between the plurality of luminescent particles through the formation of the crosslinked structure by the first substrate of the transglutaminase that the luminescent particles each have in the liquid sample through utilization of an enzyme. For example, when the target substance is the transglutaminase, the crosslinked structure between the luminescent particles is formed by causing the substance to react with the luminescent particles, and hence the luminescent particles can be aggregated. At this time, in the reaction, the contents of the liquid sample are suitably mixed at the temperature at which the transglutaminase is activated (e.g., 30° C. or more and 50° C. or less) and the pH at which the transglutaminase is activated (e.g., a pH of 5 or more and 8 or less).

In addition, when the target substance is the inhibitor of the transglutaminase serving as an example of a substance related to an enzyme reaction, an aggregation reaction between the luminescent particles can be detected by adding the transglutaminase to a solution containing the inhibitor and the luminescent particles, and causing the transglutaminase to react therewith. In this case, although the order of the reactions is not particularly limited, the inhibitor serving as the target substance can be added in advance of the transglutaminase because the transglutaminase serves as a trigger to form aggregation between the particles. The reaction solution may be divided into a plurality of solutions. That is, the following may be performed: a first solution obtained by mixing the transglutaminase and the inhibitor serving as the target substance is prepared; and then the first solution is mixed into a second solution containing the luminescent particles to perform an aggregation reaction between the luminescent particles. At this time, even when the target substance is the activator of the transglutaminase or the second substrate of the transglutaminase, the substance can be detected by performing the same operation as that in the case of the inhibitor.

### (Liquid Sample)

The liquid sample that may be used in the present disclosure may be any liquid (also referred to as “specimen”) that contains a component for measuring the activity and concentration of the target substance, and also contains the target substance. In the present disclosure, the target substance is a substance related to the enzyme reaction of the transglutaminase. For example, a body fluid, such as blood, urine, or saliva, containing the target substance, a buffer solution containing the target substance, a culture liquid or tissue extract of cells or microorganisms containing the target substance, drinking water containing the target substance, river water containing the target substance, or a waste liquid containing the target substance may be used as the specimen.

### (Transglutaminase)

The target substance in the present disclosure is, for example, a transglutaminase that is an enzyme. The transglutaminase uses a glutamine residue in a polypeptide as a substrate, and catalyzes an amide bond-forming reaction between a carboxamide side chain of the glutamine residue and an amino group that a lysine residue has. In the present disclosure, 8 enzymes/enzyme precursors referred to as “TG1” to “TG7” and “factor XIII” may each be used as the transglutaminase.

(Substance related to Enzyme Reaction)

A substance related to an enzyme reaction serving as another example of the target substance in the present disclosure is a substance related to a transglutaminase enzyme reaction, and only needs to be a substance whose amount and activity can be measured by the detection method of the present disclosure. That is, the substance related to the enzyme reaction only needs to be a substance that affects the enzyme reaction and activity of the transglutaminase. The substance is, for example, the substrate (second substrate), activator, or inhibitor of the transglutaminase. The value related to the fluorescence anisotropy obtained by the fluorescence polarization measurement of the present disclosure may be changed in accordance with the amount and activity of each of those substances related to the enzyme reaction. Examples of the second substrate of the transglutaminase include casein, gelatin, collagen, keratin, modified bodies of each thereof, and partial peptides of each thereof. In particular, a compound that is subjected to catalytic activity by the transglutaminase as compared to the first substrate of the transglutaminase is suitable. The activator is, for example, calcium chloride, and the inhibitor is, for example, a metal chelator or a protein modifier. In the present disclosure, the transglutaminase is included in the concept of the substance related to the enzyme reaction of the transglutaminase because the enzyme reaction occurs by virtue of the presence of the transglutaminase.

### (Luminescent Particle Having First Substrate of Transglutaminase)

The luminescent particle having the first substrate of the transglutaminase to be used in the present disclosure only needs to be the following luminescent particle: the particle contains a luminescent substance, and a value related to the fluorescence anisotropy of its light emission can be obtained by fluorescence polarization measurement. In addition, the luminescent particle according to the present disclosure has the first substrate of the transglutaminase, and hence a crosslinked structure can be formed between the luminescent particles by the action of the transglutaminase. The mode of crosslinking between the luminescent particles may be any bond as long as the bond is catalyzed by the transglutaminase. However, a chemical bond is exemplary, and an amide bond, a peptide bond, an isopeptide bond, or the like is also suitable. The first substrate that the luminescent particle has may be incorporated in any state as long as the substrate is recognized by the transglutaminase present in the same solution. The substrate can be present on the surface of the luminescent particle.

To form an amide bond, the luminescent particle can have, as the first substrate of the transglutaminase, at least one glutamine residue and at least one lysine residue on its surface. In this case, examples of the first substrate of the transglutaminase to be immobilized onto the luminescent particle include casein, gelatin, collagen, keratin, modified bodies of each thereof, and partial peptides of each thereof. A known method of immobilizing the transglutaminase onto a protein or peptide particle may be used as a method of immobilizing the first substrate of the transglutaminase onto the luminescent particle. Examples thereof include a physical adsorption method and a chemical bonding method, and the first substrate of the transglutaminase can be physically adsorbed to a luminescent particle having a hydrophobic surface. In addition, the first substrate of the transglutaminase can be bonded to a luminescent particle having a reactive functional group through a chemical bond. For example, an amide coupling method including using a water-soluble carbodiimide is suitable.

In the luminescent particle of the present disclosure, the average particle diameter that is the average of the diameters of the particles can be a diameter of 1 nm or more and 1,000 nm or less, exemplarily 25 nm or more and 500 nm or less, and the average particle diameter can be more specifically 50 nm or more and 300 nm or less. When the average particle diameter is more than 500 nm, there is a possibility that fluorescence anisotropy before the crosslinking between the luminescent particles increases, and hence the amount of change in value related to the fluorescence anisotropy after the aggregation between the luminescent particles reduces. In addition, when the average particle diameter is less than 25 nm, the content of the luminescent substance in the particle decreases, and the luminescence intensity per particle decreases. As a result, detection sensitivity and accuracy may decrease, and hence a luminescent particle of 50 nm or more can be used. The size of the luminescent particle may be examined by dynamic light scattering (DLS) measurement.

As a particle material (matrix material) for the luminescent particle, for example, a particle of a synthetic polymer, such as polystyrene or polymethacrylate, an inorganic particle, such as silica, titanium oxide, or iron oxide, or a natural polymer, such as dextran or casein, may be used. Of those, a synthetic polymer can be used as the particle material for the luminescent particle according to the present disclosure because the synthetic polymer has a specific gravity that allows dispersion in water, is stable, and has high productivity. A synthetic polymer is, for example, polystyrene particle that is excellent in particle size.

In addition, the surface of the particle can be hydrophilized. This is because when the surface of the particle is hydrophilized, non-specific aggregation between the luminescent particles can be suppressed, and non-specific adsorption of the luminescent particle to a measurement container (e.g., a plastic cuvette or quartz glass) can also be suppressed. In the detection method using the fluorescence polarization measurement of the present disclosure, a change in mobility of the luminescent particle irrespective of the activity and amount of an enzyme, and the concentration of a substance related to an enzyme reaction causes a reduction in accuracy or precision of the detection of the target substance. Accordingly, it is required to avoid non-specific aggregation between the luminescent particles and non-specific adsorption of the luminescent particle to the container. For this reason, the surface of the particle can be coated with a hydrophilic polymer. For example, a polymer, such as polyvinylpyrrolidone, polyethylene glycol, polyhydroxymethacrylate, or dextran, can be used. As described above, appropriate introduction of a crosslinkable functional group into the surface of the luminescent particle enables the chemical bonding of the first substrate of the transglutaminase to the particle. For example, when the surface of the luminescent particle has a reactive functional group, such as a thiol group, an amino group, a carboxy group, or a maleimide group, the first substrate of a transglutaminase having a thiol group, a carboxy group, an amino group, or the like can be bonded to the particle. The surface of the luminescent particle is, for example, a surface, which is coated with a hydrophilic polymer such as polyvinylpyrrolidone, and into which a carboxy group for chemically bonding the first substrate of the transglutaminase to the particle is introduced.

The luminescent particle to be used in the present disclosure is characterized by containing a luminescent substance. In this case, the luminescent substance may be any substance that can be incorporated into a particle and can be measured by fluorescence polarization measurement. The luminescent substance may be incorporated into the particle surface or the inside of the particle, and is exemplary incorporated into the inside of the particle. This is because an interaction between the luminescent substance and a contaminant or a water molecule in a solution may occur on the particle surface to affect the dispersibility of the luminescent particle and the obtainment of the value related to the fluorescence anisotropy. Examples of the luminescent substance include a fluorescent dye, such as a fluorescein derivative, a rhodamine derivative, or a cyanine derivative, and a rare earth luminescent complex, such as a europium complex or a terbium complex. The europium complex is suitable as the luminescent substance to be incorporated into the luminescent particle according to the present disclosure because the complex has the following features: its emission wavelength and intensity are not easily affected by the surroundings and its emission lifetime is long.

Specific examples of the europium complex include [tris(2-thenoyltrifluoroacetone)bis(triphenylphosphineoxide)europium(III)], [tris(2-thenoyltrifluoroacetone)(triphenylphosphineoxide)(dibenzylsulfoxide)europium(III)], and [tris(2-thenoyltrifluoroacetone)(phenanthroline)europium(III)].

The content of the luminescent substance in the luminescent particle can be high. This is because the luminescence intensity per particle increases, and as a result, the increase can contribute to higher sensitivity or improved measurement accuracy of the measurement of the activity and amount of an enzyme, and the concentration of a substance related to an enzyme reaction of the present disclosure. The content of the rare earth luminescent complex in the luminescent particle can be, for example, 0.001 g or more of the rare earth luminescent complex per 1 g of the particle. With this content, the luminescence intensity per particle is sufficiently high and stable luminescence can be obtained. In addition, the content of the luminescent substance may be measured by elemental analysis using the specific element of the luminescent substance as an indicator, and for example, the content of the rare earth luminescent complex may be calculated from the quantification of the rare earth luminescent complex by inductively coupled plasma (ICP) emission measurement.

### (Second Step)

In the present disclosure, the second step is a step of obtaining the value related to the fluorescence anisotropy of the liquid sample after the first step. In the present disclosure, a step of measuring a degree of fluorescence polarization or fluorescence anisotropy as an example of the value related to the fluorescence anisotropy is described. The fluorescence polarization measurement may be performed by a known method, and various commercially available measurement apparatus may be used for that purpose. The fluorescence polarization measurement is also called fluorescence depolarization measurement, but in the present specification, the fluorescence polarization measurement and the fluorescence depolarization measurement are synonymous.

In the present disclosure, an enzyme or a substance related to an enzyme reaction serving as a target substance may be subjected to fluorescence polarization measurement by using the mobility of a luminescent particle in a sample solution as an indicator. That is, the crosslinked structure between the luminescent particles is formed in the sample solution in accordance with the amount and activity of the enzyme or the substance related to the enzyme reaction, and as a result, the mobility of each of the luminescent particles reduces. The present disclosure is characterized in that the mobility of the luminescent particle is detected by the fluorescence polarization measurement. As described above, in the fluorescence polarization measurement, for example, a degree of fluorescence polarization or fluorescence anisotropy may be used as an indicator. An example in which the degree of fluorescence polarization (mp) is used as an indicator is described below.

A feature of the present disclosure is to use, as a probe, a luminescent particle containing a europium complex, which has a long emission lifetime and exhibits polarized luminescence, as a luminescent substance in the inside of the luminescent particle. A slight change in rotational movement of the luminescent particle in a liquid sample may be captured as a change in polarized luminescence characteristic. Specifically, when the crosslinking of the luminescent particles occurs between the luminescent particles to greatly reduce the mobility of each of the luminescent particles, a reduction in rotational Brownian motion of the luminescent particle may be captured with high sensitivity as a change in degree of fluorescence polarization.

The timing of the measurement of the degree of fluorescence polarization may be set appropriately. For example, the degree of fluorescence polarization may be measured after the first step. In addition, the degree of fluorescence polarization may be measured during a period from a time point in the first step to a time point after the first step. Each of the cases is a concept included in the obtainment of the value related to the fluorescence anisotropy after the first step, and may be referred to as “obtainment of the value related to the fluorescence anisotropy after the first step.” This mode is simple to operate because the degree of fluorescence polarization only needs to be measured at the time point of the completion of the reaction. For example, the degree of fluorescence polarization may be measured 5 minutes after the target substance has been added to the solution.

Alternatively, the degree of fluorescence polarization of the luminescent particle may be measured before the reaction with the target substance (enzyme or substance related to an enzyme reaction) in the first step, and the degree of fluorescence polarization may be set as an initial degree of fluorescence polarization (mp(0)), and then the reaction may proceed and a degree of fluorescence polarization (mp(t)) at a certain time point may be measured again through the crosslinking between the luminescent particles, or the degree of fluorescence polarization may be continuously measured at regular time intervals. That is, by measuring the change in degree of fluorescence polarization over time, the amount of change in degree of fluorescence polarization (Amp) may be determined from a difference between the degree of fluorescence polarization (mp(t)) at a certain time point and the initial degree of fluorescence polarization (mp(0)). The change in degree of fluorescence polarization occurs rapidly, and for example, a sufficient Amp is obtained in from about 1 minute to about 10 minutes. A rate of change in degree of fluorescence polarization (dmp/dt) may also be determined.

Measurement conditions for the degree of fluorescence polarization can be, for example, as follows: in a liquid at a temperature of from 1° C. to 50° C., the viscosity of the liquid is from 0.5 mPa·s to 50 mPa·s. When the luminescent particle is a luminescent particle containing a europium complex, the concentration of the luminescent particle is not limited as long as the luminescence of the luminescent particle can be detected, and the concentration may be appropriately selected in accordance with, for example, the kind, amount, and activity of the target substance. The degree of fluorescence polarization can be, for example, in the range of from 0.0001 mg/ml (corresponding to 0.00001% by mass) to 1.0 mg/ml (corresponding to 0.1% by mass). The degree of fluorescence polarization can be measured in the range of from 0.001 mg/ml (corresponding to 0.0001% by mass) to 0.1 mg/ml (corresponding to 0.01% by mass) to ensure the luminescence intensity from the luminescent particle and avoid the influence of the scattering of the luminescent particle.

### (Third Step)

In the present disclosure, the third step is a step of detecting a substance related to an enzyme reaction in the liquid sample based on the value related to the fluorescence anisotropy of the luminescent particle in the liquid sample obtained in the second step. More specifically, the third step is a step of associating a change in value related to the fluorescence anisotropy with the amount and activity of an enzyme or a substance related to an enzyme reaction.

In the second step, the extent to which the mobility of each of the luminescent particles reduces in accordance with the crosslinked structure between the luminescent particles is detected as a value related to the fluorescence anisotropy. The extent to which the mobility of each of the luminescent particles reduces depends on the amount and activity of the transglutaminase or the substance related to the enzyme reaction present in the liquid sample. Accordingly, the amount and activity of the transglutaminase or the substance related to the enzyme reaction in the liquid sample can be measured by associating the value related to the fluorescence anisotropy, the value being obtained in the second step, with the amount and activity of the transglutaminase or the substance related to the enzyme reaction in the liquid sample. For example, a case in which the value related to the fluorescence anisotropy, the value being obtained in the second step, is high means that the luminescent particles have aggregated and the ratio of the aggregate is high. That is, it can be concluded from the foregoing that the activity of the transglutaminase is high.

Quantitative association may be performed by, for example, determining a relational formula between the amount or activity of an enzyme and a value related to fluorescence anisotropy in advance using an enzyme solution having known enzyme activity, and then using this formula to determine the enzyme activity of the target substance from the value related to the fluorescence anisotropy obtained by detecting the target substance. When such association is performed, the enzyme activity in the liquid sample can be measured. The enzyme activity measured here may also be determined as an enzyme concentration, a mass, the number of molecules, or the like.

In addition, in the third step, the change in value related to the fluorescence anisotropy of the luminescent particle may be handled relatively. That is, the amount and activity of an enzyme or a substance related to an enzyme reaction may be associated relatively, and for example, the value of the degree of fluorescence polarization (mp) obtained by fluorescence polarization measurement may be used. For example, in applications for screening enzymes each having high activity, the screening may be performed simply based on the value of the degree of fluorescence polarization (mp) or the value of the amount of change thereof (Amp) for an enzyme population of the same concentration.

### (Reagent for Detection)

According to the present disclosure, there can be provided a reagent for use in the method of detecting a target substance related to the enzyme reaction of a transglutaminase in the present disclosure, the reagent including a luminescent particle having the first substrate of the transglutaminase.

One specific example thereof is a reagent for detecting a transglutaminase through use of a value related to fluorescence anisotropy, the reagent including a plurality of luminescent particles each having a first substrate of the transglutaminase.

Another example thereof is a reagent for detecting one of the activator of the transglutaminase or the inhibitor of the transglutaminase through use of a value related to fluorescence anisotropy, the reagent including: the transglutaminase; and the plurality of luminescent particles each having the first substrate of the transglutaminase.

Still another example thereof is a reagent for detecting the second substrate of the transglutaminase through use of a value related to fluorescence anisotropy, the reagent including: the transglutaminase; and the plurality of luminescent particles each having the first substrate of the transglutaminase. As described above, the second substrate has substrate specificity different from that of the first substrate, and has substrate specificity higher than that of the first substrate.

The luminescent particle having the first substrate of the transglutaminase, the transglutaminase, the activator of the transglutaminase, or the inhibitor of the transglutaminase for forming the reagent are each exemplary provided in a solution state. However, the materials may each be provided in, for example, a dry state, a frozen state, or a lyophilized state. The reagent in a dry state may be subjected to measurement by being brought into a solution state with a dissolving liquid attached thereto before the measurement. In addition, the reagent of the present disclosure may be a single reagent, or may be divided into a plurality of reagents, such as a first liquid, a second liquid, and a third liquid.

The amount (concentration) of the luminescent particle having the first substrate of the transglutaminase to be incorporated into the reagent in the present disclosure is, for example, from 0.000001% by mass to 20% by mass, more specifically from 0.0001% by mass to 1% by mass. With regard to the concentration of the luminescent particle described in the present disclosure, 1% by mass corresponds to 10 mg/mL. The reagent according to the present disclosure may include a substance, such as a solvent or a blocking agent, in addition to the configuration according to the present disclosure to the extent that the object of the present disclosure can be achieved.

The substances, such as a solvent and a blocking agent, may be incorporated in combination thereof. Examples of the solvent to be used in the present disclosure include various buffer solutions, such as a phosphate buffer solution, a glycine buffer solution, a Good buffer solution, a Tris buffer solution, and an ammonia buffer solution. However, the solvent to be incorporated into the reagent in the present disclosure is not limited thereto. In addition, a sensitizer that accelerates the aggregation of the luminescent particles may be incorporated into the reagent according to the present disclosure. Examples of the sensitizer include polyvinyl alcohol, polyvinylpyrrolidone, and polyalginic acid. However, the present disclosure is not limited thereto.

### (In Vitro Diagnostic Kit)

According to the present disclosure, there can also be provided an in vitro diagnostic kit including the above-mentioned reagent. The in vitro diagnostic kit may include a standard solution, a positive control, a negative control, a serum diluent, or the like in addition to the above-mentioned reagent. The standard solution is a solution of the target substance whose concentration is known. In addition to serum or physiological saline free of the target substance that may be detected, a solvent may be used as the medium of the positive control or the negative control.

### EXAMPLES

The present disclosure is described in more detail below by way of Examples.

### (Luminescent Particles S1: Production of Casein-immobilized Luminescent Particles)

The luminescent particle to be used in the method of measuring the amount and activity of an enzyme or a substance related to an enzyme reaction according to the present disclosure is characterized by having the first substrate of the transglutaminase capable of crosslinking the luminescent particles on its surface. A synthesis example of luminescent particles each having casein serving as the first substrate of the transglutaminase on its surface is described below.

First, polyvinylpyrrolidone (PVP-K30: manufactured by Tokyo Chemical Industry Co., Ltd.) was dissolved in a 2-morpholinoethanesulfonic acid (MES) buffer solution (manufactured by Kishida Chemical Co., Ltd.) having a pH of 7 to prepare a solvent A. Subsequently, [tris(2-thenoyltrifluoroacetone)bis(triphenylphosphineoxide)europium(III)] (manufactured by Central Techno Corporation, hereinafter abbreviated as “Eu(TTA)3(TPPO)2”) serving as a europium complex, a styrene monomer (manufactured by Kishida Chemical Co., Ltd.), and 3-methacryloxypropyltrimethoxysilane (manufactured by Tokyo Chemical Industry Co., Ltd., hereinafter abbreviated as “MPS”) were mixed to prepare a reaction liquid B. The reaction liquid B was added into a four-necked flask containing the solvent A, and the mixture was stirred with a mechanical stirrer set to 300 rpm. After 15 minutes of stirring under a nitrogen flow condition, the temperature of an oil bath that had been prepared was set to 70° C., and the nitrogen flow was performed for an additional 15 minutes. After the mixed liquid had been heated and stirred, an aqueous solution having dissolved therein potassium persulfate (hereinafter abbreviated as “KPS”) (manufactured by Sigma-Aldrich) was added into the reaction solution, and emulsion polymerization was performed for 20 hours. After the polymerization reaction, the resultant suspension was subjected to ultrafiltration with about 4 L of ion-exchanged water through use of an ultrafiltration membrane having a molecular weight cutoff of 100K to wash the product, to thereby provide a dispersion of luminescent particles.

An aliquot of the dispersion of the luminescent particles obtained by the emulsion polymerization was taken and added to an aqueous solution having dissolved therein 1% by mass of Tween 20 (manufactured by Kishida Chemical Co., Ltd.). After 10 minutes of stirring, a silane coupling agent, X12-1135 (manufactured by Shin-Etsu Chemical Co., Ltd.), was added, and the mixture was stirred overnight (a mass ratio among the particles, pure water, and X12-1135 loaded was 1:300:2). After the stirring, the dispersion was centrifuged, the supernatant was removed, and the precipitate was redispersed with pure water. The operations of centrifugation and redispersion were performed three or more times to wash the product. The precipitate after the washing was redispersed in pure water. Thus, luminescent particles 1 were obtained.

Next, a 0.25 mL aliquot of the particle dispersion of the luminescent particles 1 (particle concentration: 1.2% by mass) was taken, and the solvent was replaced with 1.6 mL of a MES buffer solution having a pH of 6.0. To the MES buffer solution in which the luminescent particles were dispersed, 1-[3-(dimethylamino) propyl]-3-ethylcarbodiimide and N-hydroxysulfosuccinimide sodium salt were added at 0.5% by mass, and the mixture was subjected to a reaction at 25° C. for 1 hour. After the reaction, the dispersion was washed with phosphate buffered saline (hereinafter abbreviated as “PBS”) having a pH of 7.4. Further, casein (derived from milk, manufactured by Wako Pure Chemical Industries, Ltd.) was added to the washed product, and the mixture was subjected to a reaction at 25° C. for 2 hours so that casein was introduced into the surface of each of the particles. After the introduction, the luminescent particles were washed with a Tris buffer solution having a pH of 8.

After that, the luminescent particles were washed with PBS containing 0.01% by mass Tween 20 (hereinafter abbreviated as “PBS-T”) to provide casein-immobilized luminescent particles (hereinafter referred to as “luminescent particles S1”) whose concentration was 1.0% by mass. An aqueous solution containing the luminescent particles S1 is referred to as “solution of the luminescent particles S1.” The concentration of the particles was adjusted by dilution with the PBS-T.

With regard to the fact that casein was introduced into each of the luminescent particles, the presence of casein on the surface of each of the particles was able to be recognized by: adding a BCA reagent (PROTEIN ASSAY BCA KIT, manufactured by Wako Pure Chemical Industries, Ltd.) to the solution of the luminescent particles S1; centrifuging the solution of the luminescent particles S1; then measuring the absorbance (562 nm) of the supernatant; and recognizing that the absorbance increased as compared to that before the addition of the BCA reagent. The sizes of the luminescent particles in the aqueous solution were analyzed by dynamic light scattering (DLS) measurement. As a result, the particles had an average particle diameter of 143.1 nm (z-average particle diameter). The particles had a polydispersity index of 0.019, and hence monodisperse luminescent particles were obtained.

### (Luminescent Particles S2: Production of Gelatin-immobilized Luminescent Particles)

Luminescent particles each having gelatin serving as the first substrate of the transglutaminase were synthesized in the same manner as in the method of producing the luminescent particles S1 except that casein was changed to gelatin (TYPE A, manufactured by Wako Pure Chemical Industries, Ltd.). The resultant gelatin-immobilized luminescent particles are hereinafter referred to as “luminescent particles S2.”

With regard to the fact that gelatin was introduced into each of the luminescent particles, the presence of gelatin on the surface of each of the particles was able to be recognized by: adding a BCA reagent (PROTEIN ASSAY BCA KIT, manufactured by Wako Pure Chemical Industries, Ltd.) to the solution of the luminescent particles S2; centrifuging the solution of the luminescent particles S2; then measuring the absorbance (562 nm) of the supernatant; and recognizing that the absorbance increased as compared to that before the addition of the BCA reagent. The sizes of the luminescent particles S2 in the aqueous solution were analyzed by dynamic light scattering (DLS) measurement. As a result, the particles had an average particle diameter of 151.3 nm (z-average particle diameter). The particles had a polydispersity index of 0.031, and hence monodisperse luminescent particles were obtained. The particles were increased in sizes as compared to the luminescent particles S1 (casein-immobilized luminescent particles). This is probably because of a difference between the molecular weights of the proteins (because the molecular weight of gelatin is larger than that of casein).

### (Luminescent Particles NC: Production of Tris-immobilized Luminescent Particles)

Luminescent particles each having Tris were synthesized in the same manner as in the method of producing the luminescent particles S1 except that casein was changed to tris(hydroxymethyl)aminomethane (Tris, manufactured by Wako Pure Chemical Industries, Ltd.). The resultant Tris-immobilized luminescent particles are hereinafter referred to as “luminescent particles NC.” The luminescent particles NC were each free of any compound corresponding to the first substrate of the transglutaminase, and were designed so that no aggregation between the luminescent particles was caused by the transglutaminase. In this Example, the particles were used as control particles for use in Comparative Examples.

The sizes of the luminescent particles NC in the aqueous solution were analyzed by dynamic light scattering (DLS) measurement. As a result, the particles had an average particle diameter of 141.5 nm (z-average particle diameter). The particles had a polydispersity index of 0.007, and hence monodisperse luminescent particles were obtained. The sizes of the particles were smaller than those of the luminescent particles S1 and the luminescent particles S2. This is probably because no protein is immobilized onto each of the particles.

### Example 1 and Comparative Example 1: Fluorescence Polarization Measurement of Transglutaminase with Casein-Immobilized Luminescent Particles

The PBS-T serving as a diluent and the solution of the luminescent particles S1 (particle concentration: 0.1 mg/mL) were mixed at ratios shown in Table 1 in a 96-well microplate. A solution of a transglutaminase (TG-S, 2.29 mg/mL, manufactured by Ajinomoto Co., Inc.) serving as a target substance was added to the aqueous solution, and the mixture was stirred well (Example 1). In Table 1, the transglutaminase is represented as “TG”. Casein and the transglutaminase were added after having been dissolved in water. As Comparative Example, a sample to which no target substance was added was also prepared. At this time, 10 μL of the PBS-T was added instead of the target substance (Comparative Example 1).

The microwell plate was set in a fluorescence polarization measurement apparatus (Nivo Multimode Microplate Reader). A temperature in the apparatus was adjusted to 37° C. The degree of fluorescence polarization (mp) of the sample solution was measured about 10 minutes after the addition of the target substance. The conditions of the fluorescence polarization measurement apparatus are as follows:
Mode: FP kinetics;
Excitation light: center wavelength of 355 nm/width of 40 nm;
Emission filter: (S) center wavelength of 615 nm/width of 8 nm, (P) center wavelength of 615 nm/width of 8 nm;
Dichroic mirror: D400;
Measurement time: 1,000 ms;
Z-focus: 5 mm;
Measurement spot size: 2 mm on excitation side, 4 mm on emission side;
Flash energy: low (10); and
PMT HV: 1,000.

As shown in Table 1, although the degree of fluorescence polarization of the transglutaminase-free sample (Comparative Example 1) was 60.7, the degree of fluorescence polarization of the transglutaminase-containing sample (Example 1) was as large as 63.3. The foregoing means that in Example 1, the mobility of each of the luminescent particles S1 (casein-immobilized luminescent particles) in the sample solution reduces. Thus, it was found that the aggregation of the luminescent particles S1 was caused by the activity of the transglutaminase. It was found from this Example that the transglutaminase was able to be simply and quickly detected by measuring the degree of fluorescence polarization of the sample solution containing the luminescent particles S1.

### Example 2 and Comparative Example 2: Fluorescence Polarization Measurement of Transglutaminase with Gelatin-Immobilized Luminescent Particles

The PBS-T and the solution of the luminescent particles S2 (particle concentration: 0.1 mg/mL) were mixed at ratios shown in Table 1 in a 96-well microplate, followed by fluorescence polarization measurement in the same manner as in each of Example 1 and Comparative Example 1 (Example 2 and Comparative Example 2). As can be seen from the results of the measurement shown in Table 1, although the degree of fluorescence polarization of the transglutaminase-free sample (Comparative Example 2) was 62.1, the degree of fluorescence polarization of the transglutaminase-containing sample (Example 2) was as large as 64.3. The foregoing means that in Example 2, the mobility of each of the luminescent particles S2 (gelatin-immobilized luminescent particles) in the sample solution reduces. Thus, it was found that the aggregation of the luminescent particles S2 was caused by the activity of the transglutaminase. It was found from this Example that the transglutaminase was able to be simply and quickly detected by measuring the degree of fluorescence polarization of the sample solution containing the luminescent particles S2.

When Comparative Example 1 and Comparative Example 2 that were each free of the transglutaminase were compared to each other, Comparative Example 2 had the higher degree of fluorescence polarization. The foregoing results from a difference between the particle diameters of the luminescent particles each having the first substrate of the transglutaminase. That is, this is because the luminescent particles S2 (151.3 nm) have particle diameters larger than those of the luminescent particles S1 (143.1 nm), and hence the rotational motion of each of the luminescent particles is moderate.

### Comparative Example 3 and Comparative Example 4: Control Tests with Luminescent Particles onto Each of which First Substrate is not Immobilized

The PBS-T and the solution of the luminescent particles NC (particle concentration: 0.1 mg/mL) were mixed at ratios shown in Table 1 in a 96-well microplate. The mixture was subjected to fluorescence polarization measurement in the same manner as in each of Example 1 and Comparative Example 1. As can be seen from the results of the measurement shown in Table 1, the degree of fluorescence polarization of the transglutaminase-containing sample (Comparative Example 3) was 59.4, and the degree of fluorescence polarization of the transglutaminase-free sample (Comparative Example 4) was 59.6, and hence both the samples were comparable in degree of fluorescence polarization to each other. No increase in degree of fluorescence polarization by virtue of the presence of the transglutaminase was recognized unlike Example 1 and Example 2.

TABLE 1

| | | Luminescent | Luminescent | Luminescent | TG | Degree of |
|---|---|---|---|---|---|---|
| Stock | Diluent | particles S1 | particles S2 | particles NC | (2.29 mg/mL) | fluorescence |
| solution | PBS-T | (0.1 mg/mL) | (0.1 mg/mL) | (0.1 mg/mL) | Target | polarization |
| Composition of reagent (μL) | | | | | substance (μL) | (mp) |
| Example 1 | 100 | 10 | 0 | 0 | 10 | 63.3 |
| Comparative | 100 | 10 | 0 | 0 | 0 | 60.7 |
| Example 1 | | | | | | |
| Example 2 | 100 | 0 | 10 | 0 | 10 | 64.3 |
| Comparative | 100 | 0 | 10 | 0 | 0 | 62.1 |
| Example 2 | | | | | | |
| Comparative | 100 | 0 | 0 | 10 | 10 | 59.4 |
| Example 3 | | | | | | |
| Comparative | 100 | 0 | 0 | 10 | 0 | 59.6 |
| Example 4 | | | | | | |

It was found from the results of Examples described above that the transglutaminase was able to be simply and quickly subjected to measurement by using a reagent for the fluorescence polarization detection of the transglutaminase, the reagent including, as its constituent component, a plurality of luminescent particles each having the first substrate of the transglutaminase.

The present embodiment includes the following configurations and methods.

### (Method 1)

A method of detecting a target substance related to an enzyme reaction of a transglutaminase in a liquid sample, the method comprising:
a first step of forming, in the liquid sample containing the transglutaminase and a plurality of luminescent particles each having a first substrate of the transglutaminase, a crosslinked structure of the first substrate of the transglutaminase that the plurality of luminescent particles each have based on activity of the transglutaminase to aggregate the luminescent particles;
a second step of obtaining a value related to polarization anisotropy of the liquid sample after the first step; and
a third step of detecting the target substance related to the enzyme reaction of the transglutaminase in the liquid sample based on the value related to the polarization anisotropy.

### (Method 2)

The method of detecting a target substance according to Method 1, wherein the luminescent particles are particles each containing a rare earth luminescent complex.

### (Method 3)

The method of detecting a target substance according to Method 1 or 2, wherein the first step is a step of mixing the liquid sample at 30° C. or more and 50° C. or less, and a pH of 5 or more and 8 or less.

### (Method 4)

The method of detecting a target substance according to any one of Methods 1 to 3, wherein the obtaining the value related to the polarization anisotropy in the second step is also performed in the first step.

### (Method 5)

The method of detecting a target substance according to any one of Methods 1 to 4, wherein the target substance is transglutaminase.

### (Method 6)

The method of detecting a target substance according to any one of Methods 1 to 5, wherein the target substance is a second substrate of the transglutaminase.

### (Method 7)

The method of detecting a target substance according to Method 6, wherein the second substrate has substrate specificity higher than that of the first substrate.

### (Method 8)

The method of detecting a target substance according to any one of Methods 1 to 7, wherein the target substance is an activator of the transglutaminase.

### (Method 9)

The method of detecting a target substance according to any one of Methods 1 to 8, wherein the target substance is an inhibitor of the transglutaminase.

### (Method 10)

The method of detecting a target substance according to any one of Methods 1 to 9, wherein the first substrate of the transglutaminase is selected from the group consisting of: casein; gelatin; collagen; keratin; modified bodies of each thereof; and partial peptides of each thereof.

### (Method 11)

The method of detecting a target substance according to any one of Methods 1 to 10, wherein the value related to the polarization anisotropy is one of a degree of fluorescence polarization or polarization anisotropic property.

### (Configuration 12)

A reagent for detecting a transglutaminase through use of a value related to polarization anisotropy, the reagent comprising a plurality of luminescent particles each having a first substrate of the transglutaminase.

### (Configuration 13)

A reagent for detecting one of an activator of a transglutaminase or an inhibitor of the transglutaminase through use of a value related to polarization anisotropy, the reagent comprising:
the transglutaminase; and
a plurality of luminescent particles each having a first substrate of the transglutaminase.

### (Configuration 14)

A reagent for detecting a second substrate of a transglutaminase through use of a value related to polarization anisotropy, the reagent comprising:
the transglutaminase; and
a plurality of luminescent particles each having a first substrate of the transglutaminase.

### (Configuration 15)

The reagent according to any one of Configurations 12 to 14, wherein the value related to the polarization anisotropy is one of a degree of fluorescence polarization or polarization anisotropic property.

### (Configuration 16)

The reagent according to Configuration 14, wherein the second substrate has substrate specificity higher than that of the first substrate.

### (Configuration 17)

An in vitro diagnostic kit comprising the reagent of Configuration 12 to 16.

The method of detecting a target substance related to the enzyme reaction of a transglutaminase of the present disclosure enables simple and high-sensitivity measurement of a substance related to an enzyme reaction.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-026281, filed Feb. 20, 2025, which is hereby incorporated by reference herein in its entirety.

What is claimed is:

1. A reagent for detecting a transglutaminase through use of a value related to fluorescence anisotropy, the reagent comprising a plurality of luminescent particles each having a first substrate of the transglutaminase.

2. The reagent according to claim 1, wherein the first substrate of the transglutaminase is selected from the group consisting of: casein; gelatin; collagen; keratin; modified bodies of each thereof; and partial peptides of each thereof.

3. The reagent according to claim 1, wherein the luminescent particles each contain a rare earth luminescent complex.

4. An in vitro diagnostic kit comprising the reagent of claim 1.

5. A reagent for detecting one of an activator of a transglutaminase, an inhibitor of the transglutaminase, or a second substrate of the transglutaminase through use of a value related to fluorescence anisotropy, the reagent comprising:
the transglutaminase; and
a plurality of luminescent particles each having a first substrate of the transglutaminase.

6. The reagent according to claim 5, wherein the first substrate of the transglutaminase is selected from the group consisting of: casein; gelatin; collagen; keratin; modified bodies of each thereof; and partial peptides of each thereof.

7. The reagent according to claim 5, wherein the luminescent particles each contain a rare earth luminescent complex.

8. The reagent according to claim 5,
wherein the target substance is the second substrate of the transglutaminase, and
wherein the second substrate has substrate specificity higher than that of the first substrate.

9. An in vitro diagnostic kit comprising the reagent of claim 5.

10. A method of detecting a target substance related to an enzyme reaction of a transglutaminase in a liquid sample, the method comprising:
forming, in the liquid sample containing the transglutaminase and a plurality of luminescent particles each having a first substrate of the transglutaminase, a crosslinked structure of the first substrate of the transglutaminase that the plurality of luminescent particles each have based on activity of the transglutaminase; and
obtaining a value related to fluorescence anisotropy of the liquid sample after the first step.

11. The method of detecting a target substance according to claim 10, wherein the luminescent particles are aggregated through the formation of the crosslinked structure.

12. The method of detecting a target substance according to claim 10, further comprising detecting the target substance related to the enzyme reaction of the transglutaminase in the liquid sample based on the value related to the fluorescence anisotropy.

13. The method of detecting a target substance according to claim 10, wherein the luminescent particles are particles each containing a rare earth luminescent complex.

14. The method of detecting a target substance according to claim 10, wherein forming the crosslinked structure comprises mixing the liquid sample at 30° C. or more and 50° C. or less, and a pH of 5 or more and 8 or less.

15. The method of detecting a target substance according to claim 10, wherein obtaining the value related to the fluorescence anisotropy is also performed while forming the crosslinked structure.

16. The method of detecting a target substance according to claim 10, wherein the target substance is selected from the group consisting of: the transglutaminase; an activator of the transglutaminase; and an inhibitor of the transglutaminase.

17. The method of detecting a target substance according to claim 10, wherein the target substance is a second substrate of the transglutaminase.

18. The method of detecting a target substance according to claim 17, wherein the second substrate has substrate specificity higher than that of the first substrate.

19. The method of detecting a target substance according to claim 10, wherein the first substrate of the transglutaminase is selected from the group consisting of: casein; gelatin; collagen; keratin; modified bodies of each thereof; and partial peptides of each thereof.

20. The method of detecting a target substance according to claim 10, wherein the value related to the fluorescence anisotropy is one of a degree of fluorescence polarization or a fluorescence anisotropic property.
